(19) United States
(12) Patent Application Publication
(10) Pub. No.: US 2014/0230823 A1
(43) Pub. Date: Aug. 21, 2014

(54) COLOR-CODED ENDOTRACHEAL TUBE

(71) Applicant: Cody ADAMS, Winston-Salem, NC (US)

(72) Inventor: Cody ADAMS, Winston-Salem, NC (US)

(21) Appl. No.: 14/182,356

(22) Filed: Feb. 18, 2014

Related U.S. Application Data
(60) Provisional application No. 61/766,244, filed on Feb. 19, 2013.

(51) Int. Cl. A61M 16/04

(52) U.S. Cl. CPC A61M 16/0402
USPC 128/207.14

(57) ABSTRACT

Embodiments of the present invention generally relate to endotracheal tubes having a plurality of colored markings such as colored rings therearound. The colored markings are positioned at predetermined intervals and facilitate quick and accurate determination of intubation depth. The color-coded endotracheal tubes may be accompanied by a key or legend that correlates each colored marking to a specific intubation depth.

### CROSS-REFERENCE TO RELATED APPLICATIONS

This application claims benefit of U.S. Provisional Patent Application Ser. No. 61/766,244, filed Feb. 19, 2013, which is herein incorporated by reference.

### BACKGROUND OF THE INVENTION

1. Field of the Invention

Embodiments of the present invention generally relate to medical devices, and more specifically, to endotracheal tubes.

2. Description of the Related Art

A tracheal tube is a catheter that is inserted into the trachea for the primary purpose of establishing and maintaining a patent airway and to ensure the adequate exchange of oxygen and carbon dioxide. Many different types of tracheal tubes are available, suited for different, specific applications. An endotracheal tube is a specific type of tracheal tube that is generally inserted through the mouth (orotracheal) or nose (nasotracheal).

A major patient safety issue, particularly in regards to the neonatal and pediatric populations, is self-extubation (e.g., a patient's removal of their own endotracheal tube). The self-extubations require that the patient be re-intubated. Excessive re-intubations can result in airway trauma, pulmonary compromise, and in some cases, fatality. The likelihood of self-extubations is increased when a practitioner, such as a doctor or other specialist, does not position the endotracheal tube to a proper depth. If an endotracheal tube is not positioned to a sufficient depth, extubation becomes more likely. On the other hand, if an endotracheal tube is positioned beyond the proper depth, patient trauma may occur.

Some current endotracheal tubes include numerical markings on a side thereof to represent intubation placement distances. However, these numerical markings are small and are often difficult to read, particularly in emergency situations or to inexperienced practitioners. The numerical markings are even more difficult to read on endotracheal tubes that are sized for small patients, such as infants. Thus, the numerical markings are of little use for resolving the self-extubation or over-intubation issues.

Therefore, what is needed is an endotracheal tube that allows intubation depth to be quickly and accurately determined.

### SUMMARY OF THE INVENTION

Embodiments of the present invention generally relate to endotracheal tubes having a plurality of colored markings such as colored rings therearound. The colored markings are positioned at predetermined intervals and facilitate quick and accurate determination of intubation depth. The color-coded endotracheal tubes may be accompanied by a key or legend that correlates each colored marking to a specific intubation depth.

In one embodiment, an endotracheal tube comprises a hollow tube having a plurality of colored markings therearound, the plurality of colored markings positioned at predetermined intervals to indicate an intubation depth of the endotracheal tube.

In another embodiment, an endotracheal tube comprises a hollow tube having a plurality of colored markings thereon, the plurality of colored markings each a different color and positioned at predetermined intervals to indicate an intubation depth of the endotracheal tube.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 illustrates a color-coded endotracheal tube according to one embodiment of the invention.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

Embodiments of the present invention generally relate to endotracheal tubes having a plurality of colored markings such as colored rings therearound. The colored markings are positioned at predetermined intervals and facilitate quick and accurate determination of intubation depth. The color-coded endotracheal tubes may be accompanied by a key or legend that correlates each colored marking to a specific intubation depth.

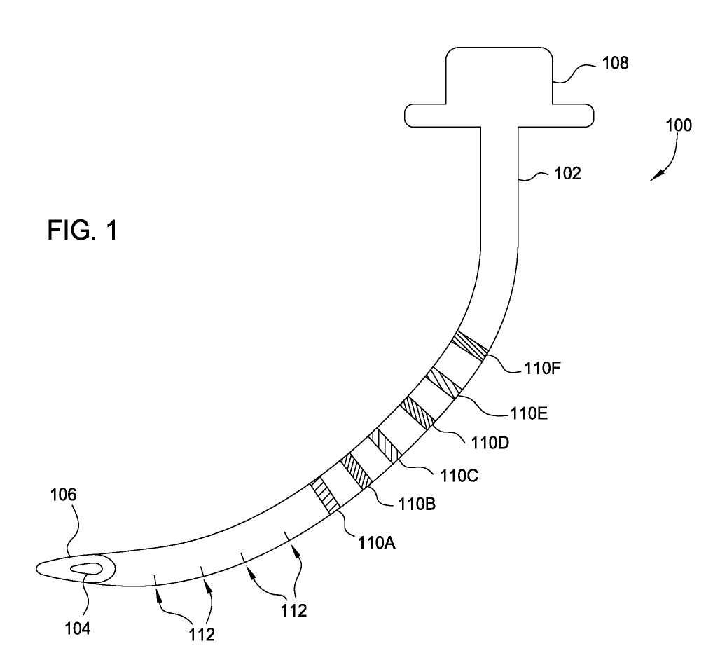

FIG. 1 illustrates a color-coded endotracheal tube 100 according to one embodiment of the invention. The color-coded endotracheal tube 100 includes a hollow tube 102 having a passage 104 therein for facilitating the flow of one or more gases, such as oxygen or carbon dioxide, therethrough. The hollow tube 102 has a curvature and includes a beveled end 106 to facilitate passage through the vocal cords of a patient. A second end of the hollow tube 102 includes a connector 108 for coupling to an external breathing system. The connector 108 may have a standard size, such as an outside diameter of about 15 millimeters (mm).

The hollow tube 102 may have a length of about 5 centimeters (cm) to about 30 cm or more, depending on patient size. Additionally, the internal and external diameters of the hollow tube 102 may be varied depending on the size (e.g., weight) of a patient. In one example, the hollow tube may have an internal diameter of about 2.5 millimeters to about 4.5 millimeters for an infant patient having a weight of about 1 kilogram (kg) to about 5 kg. The color-coded endotracheal tube 100 may be formed from a plastic or rubber, such as polyvinyl chloride. However, additional materials, such as non-toxic materials, are also contemplated.

The hollow tube 102 of the color-coded endotracheal tube 100 includes a plurality of colored markings, such as colored rings 110A-110F, visible at predetermined intervals along the outer surface of the hollow tube 102. Different colors are indicated in FIG. 1 through the use of different hatch patterns. In one example, the predetermined interval is equal to 1 cm, although, other intervals are also contemplated. The plurality of colored rings 110A-110F may be formed using a dyed material, a printed ink, a stain, or any other method of coloring the hollow tube 102. Each colored ring of the plurality of colored rings 110A-110F is positioned a predetermined distance from the beveled end 106 to facilitate accurate determination of the intubation depth of the hollow tube 102. For example, the colored ring 110A may be positioned a distance, such as 5 cm, from the beveled end 106. When the colored ring 110A is positioned adjacent to a physical marker on the patient, such as the patient's front teeth, or a corner of the patient's mouth, a practitioner can quickly and accurately determine that the endotracheal tube is intubated to the marked depth, e.g., 5 cm. Each of the plurality of colored rings 110A-110F may be a different color (or multiple colors, such as a two-tone ring) to allow a user to quickly distinguish between each of the rings 110A-110F. It is contemplated that some colors may be used more than once when located in a position that is unlikely to cause confusion to a practitioner placing the endotracheal tube 100. In addition, while six colored rings are shown, it is contemplated that the hollow tube 102 may include more or less than six colored rings or markings.

The hollow tube 102 optionally includes a plurality of marks 112, such as indentations, raised bumps, or printed marks (such as numbers), adjacent to the colored rings 110A-110F. The marks 112 are located below the minimum intubation depth, and thus, are generally not used for establishing a proper intubation depth. Because the marks 112 are not used for establishing proper intubation depth, the marks 112 do not need to be easily visible and distinguishable like the colored rings 110A-110F. The physical marks 112 are spaced apart at the same interval as the colored rings 110A-110F, and are formed on the hollow tube 102 to illustrate physical spacing on the hollow tube 102, when desired. In one example, the marks 112 indicate intubation depths outside the recommended range. In such an example, the marks 112 are included for reference purposes. Additionally or alternatively, it is contemplated that one or more colored rings 110A-110F may include a numerical marking adjacent thereto in order to numerically indicate on the endotracheal tube 100 the intubation depth of the colored rings 110A-110F.

TABLE 1

| Patient Weight | Depth | Ring | Tube Inside Dia. |
|---|---|---|---|
| (Kg) | (cm) | Color | (mm) |
| 0.5 | 6 | Yellow | 2.5 |
| 1 | 7 | Blue | 2.5 or 3.0 |
| 1.5 | 7.5 | | 3.0 |
| 2 | 8 | Green | 3.0 |
| 2.5 | 8.5 | | 3.0 |
| 3 | 9 | Purple | 3.0 |
| 3.5 | 9.5 | | 3.5 |
| 4 | 10 | Red | 3.5 or 4.0 |
| 4.5 | 10.5 | | 4.0 or 4.5 |
| 5 | 11 | Black | 4.5 |

Table 1 is a table correlating ring color to intubation depth. Table 1 may be utilized by a practitioner to determine the proper intubation depth for a patient, which size (e.g., internal diameter) endotracheal tube to use for a patient, and the correlation between intubation depth and ring color. Table 1 may optionally be included on or with the packaging for a color-coded endotracheal tube, such as color-coded endotracheal tube 100. In one example, a patient weighing approximately 0.5 kg should be intubated with an endotracheal tube having a hollow tube with an internal diameter of 2.5 mm. The patient should be intubated to a depth of 6 cm, which corresponds to a yellow ring disposed around the hollow tube.

In another example, a patient weighing about 1 kg should be intubated with an endotracheal tube having a hollow tube with an internal diameter of 2.5 mm or 3.0 mm. The patient should be intubated to a depth of 7 cm, which corresponds to a blue ring disposed around the hollow tube. In yet another example, a patient weighing about 1.5 kg should be intubated with an endotracheal tube having a hollow tube with an internal diameter of 3.0 mm. The patient should be intubated to a depth of 7.5 cm, which corresponds to a location between a blue ring and a green ring. Thus, as illustrated, the utilization of colored rings allows for quick and accurate intubation to a desired depth using the colored rings as guides, and, optionally, by interpolating therebetween. It is contemplated that the rings may be spaced at closer intervals, such as about every half centimeter, thereby reducing the occurrence of interpolation. Additionally, it is to be understood that the information in Table 1 is shown for explanation purposes, and additional information, including information for adult patients, may also be included in addition to or in place of the information shown. Several additional examples are shown in Table 1 for reference.

It is to be understood that while examples herein generally refer to colored rings being used as visual indicators of intubation depth, other markings are also contemplated as visual indicators. For example, colored lines or colored semicircles are also contemplated. Thus, it is not necessary that the visual indicators be complete rings; however, complete rings may be advantageous in some circumstances, as the complete rings are generally visible around 360 degrees of the endotracheal tube.

Benefits of the embodiments described herein generally include the quick and accurate placement of endotracheal tubes, particular in neonatal patients where conventional markings on endotracheal tubes may be difficult to read (e.g., due to unrestrained movement of a patient, or the size of the markings on a relatively smaller endotracheal tube). Because the endotracheal tubes described herein can be placed at the proper depth using colored rings as a guide, the number of re-intubations is reduced. The reduction in reintubations reduces the trauma to the airway of a patient. Moreover, the colored rings further reduce the likelihood of placing an endotracheal tube too far within a patient's airway, and thus, the occurrence of patient trauma due to bronchial intubation is also reduced.

While the foregoing is directed to embodiments of the present invention, other and further embodiments of the invention may be devised without departing from the basic scope thereof, and the scope thereof is determined by the claims that follow.

What is claimed is:

1. An endotracheal tube, comprising:
a hollow tube having a plurality of colored markings therearound, the plurality of colored markings positioned at predetermined intervals to indicate an intubation depth of the endotracheal tube.

2. The endotracheal tube of claim 1, wherein the hollow tube comprises polyvinyl chloride.

3. The endotracheal tube of claim 1, wherein the plurality of colored markings comprise dyed or printed material.

4. The endotracheal tube of claim 1, wherein the predetermined interval is about one centimeter.

5. The endotracheal tube of claim 1, wherein the predetermined interval is about 0.5 centimeters.

6. The endotracheal tube of claim 1, further comprising a plurality of marks indicating intubation depths.

7. The endotracheal tube of claim 6, wherein the marks indicate intubation depths outside of a recommended range.

8. The endotracheal tube of claim 1, wherein the endotracheal tube is curved and includes a beveled end.

9. The endotracheal tube of claim 1, wherein the colored markings are colored rings.

10. The endotracheal tube of claim 2, wherein the predetermined interval is about one centimeter.

11. The endotracheal tube of claim 2, wherein the predetermined interval is about 0.5 centimeters.

12. The endotracheal tube of claim 2, further comprising a plurality of marks indicating intubation depths.

13. The endotracheal tube of claim 12, wherein the marks include physically raised bumps.

14. The endotracheal tube of claim 12, wherein the marks include printed numbers.

15. The endotracheal tube of claim 2, wherein the endotracheal tube is curved and includes a beveled end.

16. The endotracheal tube of claim 3, further comprising a plurality of marks indicating intubation depths.

17. The endotracheal tube of claim 16, wherein the marks include physically raised bumps.

18. The endotracheal tube of claim 16, wherein the marks include printed numbers.

19. An endotracheal tube, comprising:
a hollow tube having a plurality of colored markings thereon, the plurality of colored markings each a different color and positioned at predetermined intervals to indicate an intubation depth of the endotracheal tube.

20. The endotracheal tube of claim 19, wherein the colored markings are colored rings.
